(19) United States
(12) Patent Application Publication
(10) Pub. No.: US 2020/0352547 A1
(43) Pub. Date: Nov. 12, 2020

(54) ULTRASONIC PULMONARY ASSESSMENT

(71) Applicant: KONINKLIJKE PHILIPS N.V., EINDHOVEN (NL)

(72) Inventors: Balasundar Iyyavu RAJU, NORTH ANDOVER, MA (US); Jingping XU, SHANGHAI (CN); Seungsoo KIM, ANDOVER, MA (US)

(21) Appl. No.: 16/765,357

(22) PCT Filed: Nov. 20, 2018
(86) PCT No.: PCT/EP2018/081859

§ 371 Date: May 19, 2020

Related U.S. Application Data
(60) Provisional application No. 62/589,709, filed on Nov. 22, 2017.

(30) Foreign Application Priority Data
Aug. 3, 2018 (CN) PCT/CN2018/098631

(51) Int. Cl. A61B 8/08; A61B 8/00

(52) U.S. Cl. CPC A61B 8/5269; A61B 8/4254; A61B 8/5223; A61B 8/085; A61B 8/461; A61B 8/468

(57) ABSTRACT

The present disclosure describes an ultrasound system configured to identify and evaluate B-lines that may appear during an ultrasound scan of a chest region of a subject. In some examples, the system may include an ultrasound transducer configured to acquire echo signals responsive to ultrasound pulses transmitted toward a target region comprising one or both lungs. The system can also include one or more processors communicatively coupled with the ultrasound transducer and configured to identify one or more B-lines within the target region during a scan thereof. Based on the identified B-lines, the processors can determine a severity value of the B-lines and a pulmonary diagnosis based on the severity value in substantially real time during the ultrasound scan. The diagnosis may embody a distinction between cardiogenic and non-cardiogenic pulmonary edema.

### TECHNICAL FIELD

The present disclosure pertains to ultrasound systems and methods for evaluating sonographic B-lines in a pulmonary region of a patient. Particular implementations involve systems configured to distinguish cardiogenic from non-cardiogenic causes of pulmonary edema by determining the severity and spatial distribution of B-lines during an ultrasound scan.

### BACKGROUND

Lung ultrasound can be performed by positioning an ultrasound transducer both longitudinally, perpendicular to the ribs, and obliquely, along the intercostal spaces. Among the various features evaluated via lung ultrasound to diagnose conditions such as pneumothorax (“PTX”), pneumonia, pulmonary edema and others, are visual artifacts known as B-lines. B-lines are discrete/fused vertical hyperechoic reverberations that typically extend downward, e.g., closer to maximum imaging depth, from the pleural line, which marks the interface between the chest wall and the lung.

Determining the number and spatial distribution of B-lines can be especially critical in determining the cause of pulmonary edema. In particular, the presence of B-lines may be indicative of cardiogenic pulmonary edema or non-cardiogenic pulmonary edema, but the spatial distribution of the B-lines may strongly indicate one type versus the other. Because the treatment of pulmonary edema depends largely on its etiology, identifying the spatial characteristics of B-lines can significantly impact patient outcomes. Ultrasound systems configured to accurately characterize B-lines detected during a patient scan are needed to reduce user error and improve pulmonary diagnosis.

### SUMMARY

Provided herein are ultrasound systems and methods for automatic B-line characterization. Disclosed systems can be configured to distinguish cardiogenic causes of pulmonary edema, such as heart failure, from non-cardiogenic causes, such as pneumonia. Although examples discussed herein are specific to pulmonary edema diagnosis, the systems and methods disclosed may be applied to a variety of medical assessments that depend at least in part on B-line detection and/or characterization. In various embodiments, the system can continuously detect the presence and/or severity of sonographic B-lines in substantially real time as an ultrasound transducer is moved along an imaging plane. The distance covered by the transducer can be computed using image correlation techniques, for example, or via an inertial motion sensor such as an accelerometer included in the system. The distribution of B-lines over a distance spanned by the transducer can then be automatically determined by the system. Based on the spatial distribution, the system can pinpoint the cause of pulmonary edema. For example, if the B-line pattern is diffuse, widespread and/or bilateral (present in both lungs), the system may indicate a high likelihood of cardiogenic causation. By contrast, if the B-line pattern is localized or patchy, the system may indicate a low-likelihood of cardiogenic causation. Some configurations of the system may be equipped to characterize additional features indicative of pulmonary edema etiology, such as the regularity of the pleural line. The system can be configured to present B-line information in various formats for additional user assessment.

In accordance with examples of the present disclosure, an ultrasound system may include an ultrasound transducer configured to acquire echo signals responsive to ultrasound pulses transmitted toward a target region comprising a lung. The system may also include one or more processors in communication with the ultrasound transducer and configured to identify one or more B-lines within the target region during a scan of the target region, determine a severity value of the B-lines in the target region, and determine a diagnosis based at least in part on the severity value of the B-lines.

In some examples, the processors may be configured to determine the severity value of the B-lines by determining a total number of B-lines. In some embodiments, the processors may be configured to determine the severity value of the B-lines by determining a spatial distribution of the B-lines. In some implementations, the processors may be configured to determine the spatial distribution of the B-lines within one or more sub-regions of the target region. In some examples, each of the one or more sub-regions may comprise an intercostal space such that a severity value is determined for each intercostal space within the target region. In some embodiments, the processors may be configured to determine the spatial distribution by determining a distance covered by the ultrasound transducer during the scan of the target region and dividing the distance by a total number of B-lines identified.

In some implementations, the system may also include a graphical user interface configured to display an ultrasound image from at least one image frame generated from the ultrasound echoes. According to such examples, the processors may be further configured to cause the graphical user interface to display an annotated ultrasound image in which the B-lines are labeled. In addition or alternatively, the processors may be further configured to cause the graphical user interface to display a graphical representation of the severity value of the B-lines in the target region. In some examples, the system may also include an accelerometer configured to determine a distance covered by the ultrasound transducer during the scan of the target region. In some embodiments, the diagnosis may be of cardiogenic pulmonary edema or non-cardiogenic pulmonary edema, which the processors can be configured to distinguish between by applying a threshold to the severity value.

In accordance with examples of the present disclosure, a method may involve acquiring echo signals responsive to ultrasound pulses transmitted toward a target region comprising a lung, identifying one or more B-lines within the target region during a scan of the target region, determining a severity value of the B-lines in the target region, and determining a diagnosis based at least in part on the severity value of the B-lines.

In some embodiments, determining the severity value of the B-lines may involve determining a total number of B-lines and/or a spatial distribution of the B-lines. In some implementations, determining the spatial distribution of the B-lines may involve determining a distance covered by the ultrasound transducer during the scan of the target region and dividing the distance by a total number of B-lines identified. Examples may also involve displaying an ultrasound image from at least one image frame generated from the ultrasound echoes. Embodiments may also involve displaying a graphical representation of the severity value of the B-lines in the target region and/or labeling the B-lines. In some implementations, the diagnosis comprises cardiogenic pulmonary edema or non-cardiogenic pulmonary edema. Example methods may further involve distinguishing between cardiogenic pulmonary edema and non-cardiogenic pulmonary edema by applying a threshold to the severity value.

Any of the methods described herein, or steps thereof, may be embodied in non-transitory computer-readable medium comprising executable instructions, which when executed may cause a processor of a medical imaging system to perform the method or steps embodied herein.

### BRIEF DESCRIPTION OF THE DRAWINGS

FIG. 1 is a lung ultrasound image taken with an ultrasound probe in accordance with the principles of the present disclosure;

FIG. 2 is a block diagram of an ultrasound system configured in accordance with principles of the present disclosure;

FIG. 3 is a representation of an ultrasound scan performed on a patient in accordance with principles of the present disclosure;

FIG. 4A is a diagram showing zonal B-line characterization that may be displayed on a user interface in accordance with principles of the present disclosure;

FIG. 4B is an ultrasound image displayed on a user interface in accordance with principles of the present disclosure;

FIG. 5 is a block diagram of an ultrasound method implemented in accordance with principles of the present disclosure.

### DETAILED DESCRIPTION

The following description of certain embodiments is merely exemplary in nature and is in no way intended to limit the invention or its applications or uses. In the following detailed description of embodiments of the present systems and methods, reference is made to the accompanying drawings which form a part hereof, and in which are shown by way of illustration specific embodiments in which the described systems and methods may be practiced. These embodiments are described in sufficient detail to enable those skilled in the art to practice the presently disclosed systems and methods, and it is to be understood that other embodiments may be utilized and that structural and logical changes may be made without departing from the spirit and scope of the present system. Moreover, for the purpose of clarity, detailed descriptions of certain features will not be discussed when they would be apparent to those with skill in the art so as not to obscure the description of the present system. The following detailed description is therefore not to be taken in a limiting sense, and the scope of the present system is defined only by the appended claims.

The present technology is also described below with reference to block diagrams and/or flowchart illustrations of methods, apparatus (systems) and/or computer program products according to the present embodiments. It is understood that blocks of the block diagrams and/or flowchart illustrations, and combinations of blocks in the block diagrams and/or flowchart illustrations, may be implemented by computer executable instructions. These computer executable instructions may be provided to a processor, controller or controlling unit of a general purpose computer, special purpose computer, and/or other programmable data processing apparatus to produce a machine, such that the instructions, which execute via the processor of the computer and/or other programmable data processing apparatus, create means for implementing the functions/acts specified in the block diagrams and/or flowchart block or blocks.

An ultrasound system according to the present disclosure may utilize various neural networks, for example a deep neural network (DNN), a convolutional neural network (CNN), a recurrent neural network (RNN), an autoencoder neural network, or the like, to distinguish between cardiogenic and non-cardiogenic pulmonary edema based on the number and/or distribution of B-lines detected via ultrasound imaging. In various examples, a neural network can be trained using any of a variety of currently known or later developed learning techniques to obtain a neural network (e.g., a trained algorithm or hardware-based system of nodes) that is configured to analyze input data in the form of ultrasound image frames.

An ultrasound system in accordance with principles of the present invention may include or be operatively coupled to an ultrasound transducer configured to transmit ultrasound pulses toward a medium, e.g., a human body or specific portions thereof, and generate echo signals responsive to the ultrasound pulses. The ultrasound system may include a beamformer configured to perform transmit and/or receive beamforming, and a display configured to display, in some examples, ultrasound images generated by the ultrasound imaging system. The ultrasound imaging system may include one or more processors and in some examples, at least one neural network, which may be implemented in hardware and/or software components.

The neural network implemented according to the present disclosure may be hardware- (e.g., neurons are represented by physical components) or software-based (e.g., neurons and pathways implemented in a software application), and can use a variety of topologies and learning algorithms for training the neural network to produce the desired output. For example, a software-based neural network may be implemented using a processor (e.g., single or multi-core CPU, a single GPU or GPU cluster, or multiple processors arranged for parallel processing) configured to execute instructions, which may be stored in computer readable medium, and which when executed cause the processor to perform a trained algorithm for assessing B-lines present within an ultrasound image. The ultrasound system may include a display or graphics processor, which is operable to arrange the ultrasound images and/or additional graphical information, which may include annotations, confidence metrics, user instructions, tissue information, patient information, indicators, and other graphical components, in a display window for display on a user interface of the ultrasound system. In some embodiments, the ultrasound images and associated measurements may be provided to a storage and/or memory device, such as a picture archiving and communication system (PACS) for reporting purposes or future training (e.g., to continue to enhance the performance of the neural network).

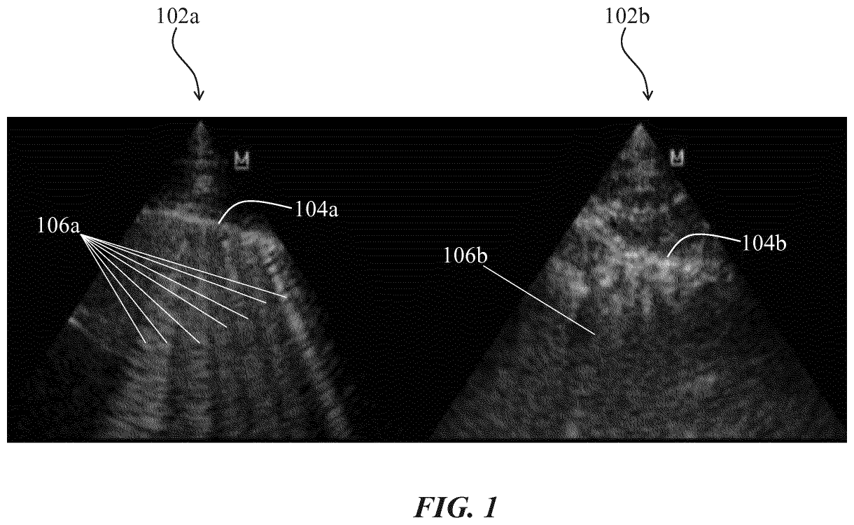

FIG. 1 includes an ultrasound image 102a indicative of cardiogenic pulmonary edema, and an ultrasound image 102b indicative of non-cardiogenic pulmonary edema, both images obtained from an article authored by P. A. Blanco and T. F. Cianciulli that is titled “Pulmonary edema assessed by ultrasound: Impact in cardiology and intensive care practice” (Echocardiography, 2016, Vol. 33:778-787). As shown, image 102a includes a distinct pleural line 104a and a plurality of uniformly distributed vertical B-lines 106a. By contrast, image 102b includes a thickened pleural line 104b and only one readily discernable B-line 106b of appreciable length. While the specific number of B-lines can vary from patient to patient, the general B-line patterns shown in FIG. 1 may be representative of cardiogenic and non-cardiogenic cases of pulmonary edema. In particular, cardiogenic pulmonary edema may be characterized by a greater number of B-lines relative to cases of non-cardiogenic pulmonary edema, which may also be indicated by a thickened pleural line. In some examples, non-cardiogenic pulmonary edema may be evidenced by patchy, localized clusters of B-lines, such that one or more portions of an associated ultrasound image may include at least one B-line, while other portions of the same image may contain zero B-lines.

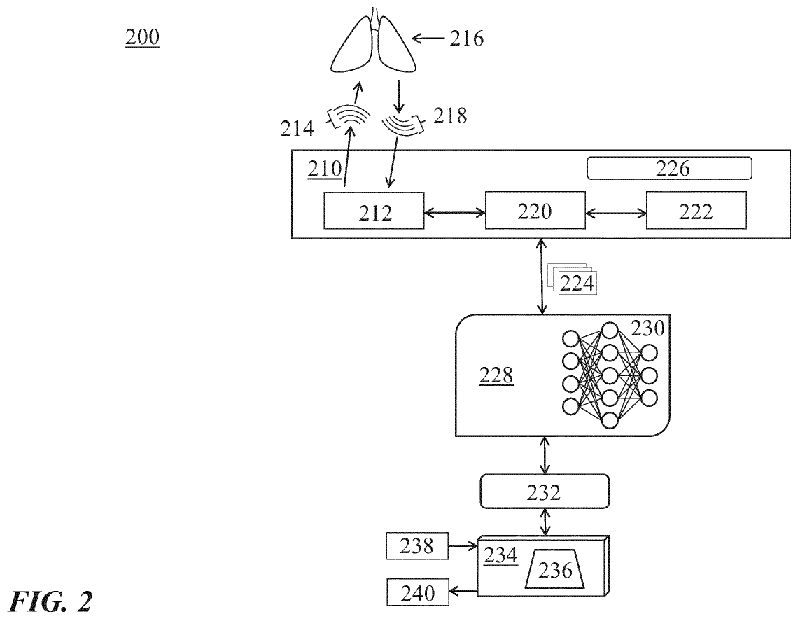

FIG. 2 shows an example ultrasound system 200 configured to identify and characterize B-lines in accordance with the present disclosure. As shown, the system 200 may include an ultrasound data acquisition unit 210. The ultrasound data acquisition unit 210 can include an ultrasound probe which includes an ultrasound sensor array 212 configured to transmit ultrasound pulses 214 into a target region 216 of a patient, which may include one or both lungs, and receive ultrasound echoes 218 responsive to the transmitted pulses. As further shown, the ultrasound data acquisition unit 210 can include a beamformer 220 and a signal processor 222, which can be configured to generate a stream of discrete ultrasound image frames 224 from the ultrasound echoes 218 received at the array 212. To monitor scan distance, the data acquisition unit 210 can also include a sensor 226 in some embodiments. The image frames 224 generated by the signal processor 222 can be communicated to a data processor 228, e.g., a computational module or circuitry, configured to determine movement of the acquisition unit 210, either alone and/or via the sensor 226, and determine the presence and/or severity of B-lines present within one or more image frames 224. Based on the B-line assessment, the data processor 228 may be configured to further determine the likelihood that cardiogenic factors caused the pulmonary edema of the patient. In some examples, the data processor 228 may be configured to implement at least one neural network, such as neural network 230, trained to assess B-line patterns and determine whether the assessed patterns indicate cardiogenic or non-cardiogenic etiology.

Determinations made by the data processor 228 can be communicated to a display processor 232 coupled with a graphical user interface 234. The display processor 232 can be configured to generate ultrasound images 236 from the image frames 224, which can then be displayed in real time on the user interface 234 as an ultrasound scan is being performed. The user interface 234 can be configured to receive user input 238 at any time before, during or after an ultrasound procedure. In addition to the displayed ultrasound images 236, the user interface 234 can be configured to generate one or more additional outputs 240, which can include an assortment of graphics displayed concurrently with, e.g., overlaid on, the ultrasound images 236. The graphics may label certain anatomical features and measurements identified by the system, such as the presence, number, location and/or spatial distribution of B-lines, an etiology notification based on the B-line determination(s), and/or indications of various organs, bones, tissues and/or interfaces, such as the pleural line. In some embodiments, the B-line(s) can be highlighted to facilitate user interpretation of the images 236. The number and/or severity of the B-lines can also be displayed, and in some examples, grouped into localized zones. Additional outputs 240 can also include annotations, confidence metrics, user instructions, tissue information, patient information, indicators, user operating instructions, and other graphic components.

The configuration of system 200 may vary. For instance, the system can be portable or stationary. Various portable devices, e.g., laptops, tablets, smart phones, or the like, may be used to implement one or more functions of the system 200. In examples that incorporate such devices, the ultrasound sensor array may be connectable via a USB interface, for example. In some embodiments, the image frames 224 generated by the data acquisition unit 210 may not be displayed. According to such embodiments, the determinations made by the data processor 228 may be communicated to a user, via the graphical user interface 234 or otherwise, in graphical and/or numerical format. In various examples, the system 200 may be implemented at the point of care, which may include emergency and critical care settings.

The ultrasound sensor array 212 may include at least one transducer array configured to transmit and receive ultrasonic energy. The settings of the ultrasound sensor array 212 can be preset for performing a particular scan, but can also be adjustable during the scan. A variety of transducer arrays may be used, e.g., linear arrays, convex arrays, or phased arrays. The number and arrangement of transducer elements included in the sensor array 212 may vary in different examples. For instance, the ultrasound sensor array 212 may include a 1D or 2D array of transducer elements, corresponding to linear array and matrix array probes, respectively. The 2D matrix arrays may be configured to scan electronically in both the elevational and azimuth dimensions (via phased array beamforming) for 2D or 3D imaging. In addition to B-mode imaging, imaging modalities implemented according to the disclosures herein can also include shear-wave and/or Doppler, for example. A variety of users may handle and operate the ultrasound data acquisition unit 210 to perform the methods described herein, including under-trained or novice users inexperienced in sonography and/or B-line assessment. Preexisting methods of pulmonary edema etiology identification depended on visual assessment, which required considerable expertise and often an extended period of evaluation time. System 200 may eliminate or at least substantially reduce the need for user interpretation to determine the causal factor(s) driving a given case of pulmonary edema, thereby decreasing the processing time needed to make causal determinations and increasing the accuracy of such determinations. Accordingly, system 200 can increase the accuracy of B-line assessment, especially for inexperienced users, and streamline the workflow for evaluating pulmonary ultrasound data.

The beamformer 220 coupled to the ultrasound sensor array 212 can comprise a microbeamformer or a combination of a microbeamformer and a main beamformer. The beamformer 220 may control the transmission of ultrasonic energy, for example by forming ultrasonic pulses into focused beams. The beamformer 220 may also be configured to control the reception of ultrasound signals such that discernable image data may be produced and processed with the aid of other system components. The role of the beamformer 220 may vary in different ultrasound probe varieties. In some embodiments, the beamformer 220 may comprise two separate beamformers: a transmit beamformer configured to receive and process pulsed sequences of ultrasonic energy for transmission into a subject, and a separate receive beamformer configured to amplify, delay and/or sum received ultrasound echo signals. In some embodiments, the beamformer 220 may include a microbeamformer operating on groups of sensor elements for both transmit and receive beamforming, coupled to a main beamformer which operates on the group inputs and outputs for both transmit and receive beamforming, respectively.

The signal processor 222 may be communicatively, operatively and/or physically coupled with the sensor array 212 and/or the beamformer 220. In the example shown in FIG. 2, the signal processor 222 is included as an integral component of the data acquisition unit 210, but in other examples, the signal processor 222 may be a separate component. In some examples, the signal processor may be housed with the sensor array 212 or it may be physically separate but communicatively (e.g., via a wired or wireless connection) coupled thereto. The signal processor 222 may be configured to receive unfiltered and disorganized ultrasound data embodying the ultrasound echoes 218 received at the sensor array 212. From this data, the signal processor 222 may continuously generate ultrasound image frames 224 as a user scans the target region 216. In some embodiments, ultrasound data received and processed by the data acquisition unit 210 can be utilized by one or more components of system 200 prior to generating ultrasound image frames therefrom.

The data processor 228 can be configured to characterize B-lines appearing in one or more image frames 224 in accordance with various methodologies. In some examples, the data processor 228 can be configured to identify B-lines by first locating the pleural line, then defining a region of interest below the pleural line and identifying B-lines from B-line candidates based on at least one imaging parameter, such as the intensity and/or uniformity of the candidates, as described for example in the U.S. Patent Application titled “Detection, Presentation and Reporting of B-lines in Lung Ultrasound” to Balasunder, R. et al., which is incorporated by reference in its entirety herein.

The data processor 228 can determine the total number of B-lines present within the target region and/or the location of one or more B-lines. For example, the data processor 228 can be configured to determine whether B-lines appear in the right anterior axillary space, or whether B-lines appear in one or more regions defined by a user.

The data processor 228 can also be configured to identify movement of the probe 210 as the probe is moved along an imaging plane, continuously determining the presence and/or severity of the identified B-lines as the probe is moved. In some embodiments, the data processor 228 can also identify the pleural line and any abnormalities thereof, for example by determining the thickness and/or continuity of the pleural line as the probe is being moved, for example as described in the U.S. Patent Application titled “Target Probe Placement For Lung Ultrasound” to Balasundar, R. et al., which is incorporated by reference in its entirety herein. Such determinations may be utilized by the data processor 228 to further inform a determination of whether pulmonary edema is caused by cardiogenic or non-cardiogenic factors. In addition or alternatively, the data processor 228 may be configured to determine one or more cardiac parameters, e.g., ejection fraction, to strengthen the B-line evaluation.

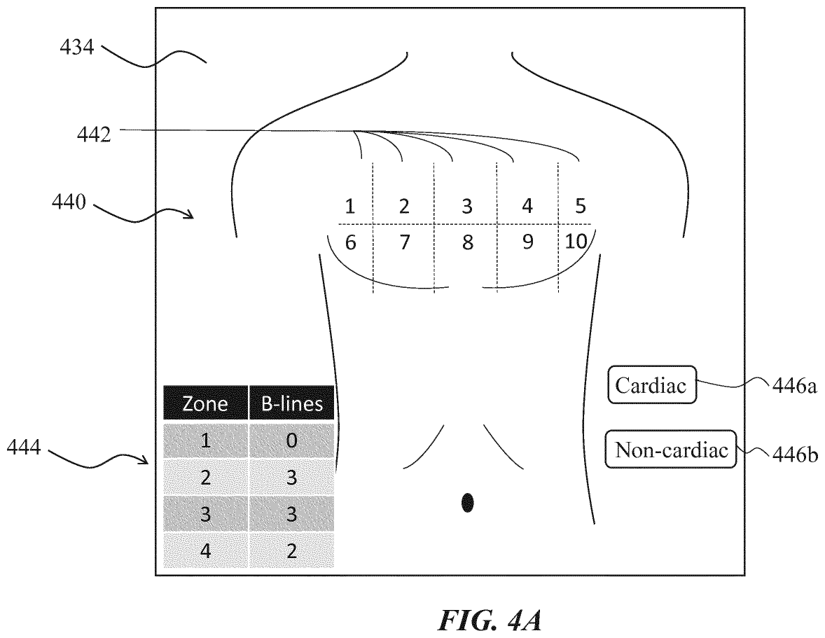

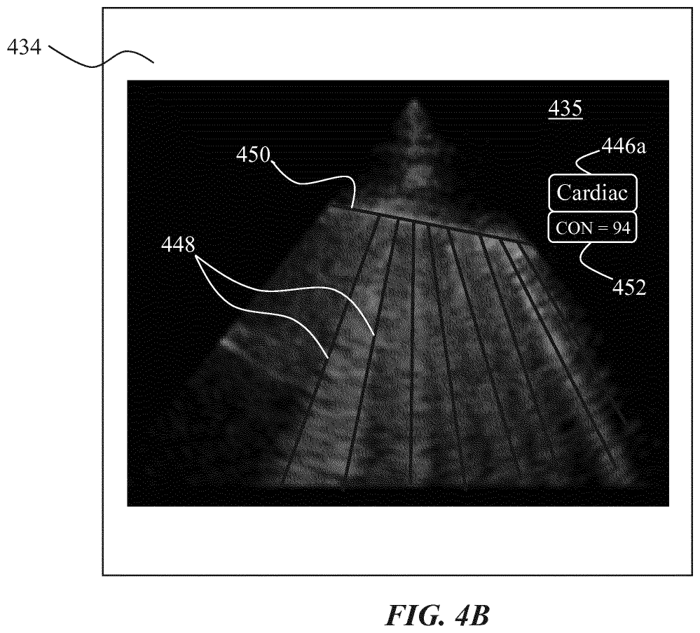

Using the number of confirmed B-lines and the lateral anatomical distance over which the B-lines were detected, the data processor 228 can then determine the spatial distribution of the B-lines, thereby also determining whether the distribution is localized or spatially diffuse. In an example, the data processor 228 can determine the spatial distribution of B-lines by dividing the total distance traversed during a scan by the total number of B-lines detected. As described further below with respect to FIG. 4A, the data processor 228 can also be configured to determine B-line distribution in various zones across a patient's chest. For example, the data processor 228 may be configured to determine the number of B-lines present within an area defined by a user, or within a default area, such as one or more intercostal spaces. The B-line severity, evidenced by the total number and/or distribution of B-lines, may then be used by the data processor 228 to estimate the likelihood that a current case of pulmonary edema is caused by cardiogenic or non-cardiogenic factors. For instance, the data processor 228 may determine that cardiogenic pulmonary edema is likely due to a moderate to high number of detected B-lines, especially if the B-lines are substantially uniformly present across the target region, as opposed to being localized in one sub-region thereof.

As mentioned above, some examples of the data processor 228 may be configured to implement a neural network 230 configured to determine whether a particular case of pulmonary edema is cardiogenic or non-cardiogenic. According to such examples, the neural network 230 may be a feed-forward neural network trained using a plurality, e.g., thousands, of ultrasound images containing various numbers and spatial distributions of B-lines. The images may be annotated according to etiology, such that images with patchy B-lines are labeled “non-cardiogenic,” and images with a high number of uniformly distributed, diffuse B-lines are labeled “cardiogenic.” The neural network 230 may continue to learn over time by periodically, e.g., with every ultrasound scan performed by system 200, inputting additional image frames 224 into the network, along with annotations of the determined etiology. By learning from a large number of annotated images, the neural network 230 may determine etiology estimates qualitatively. As such, the neural network 230 may be used to substantiate one or more numerical B-line determinations made by the data processor 228. For example, the neural network 230 may determine that a particular spatial pattern of B-lines is indicative of a high likelihood of cardiogenic pulmonary edema. The data processor 228 may determine, independently of the neural network 230, that a low total number of B-lines is indicative of a low likelihood of cardiogenic pulmonary edema. As a result, the data processor 228 may generate a notification relaying the discrepancy to a user, who may then visually examine one or more ultrasound images generated by the system. Such a discrepancy may lower a confidence metric associated with a particular etiology estimate.

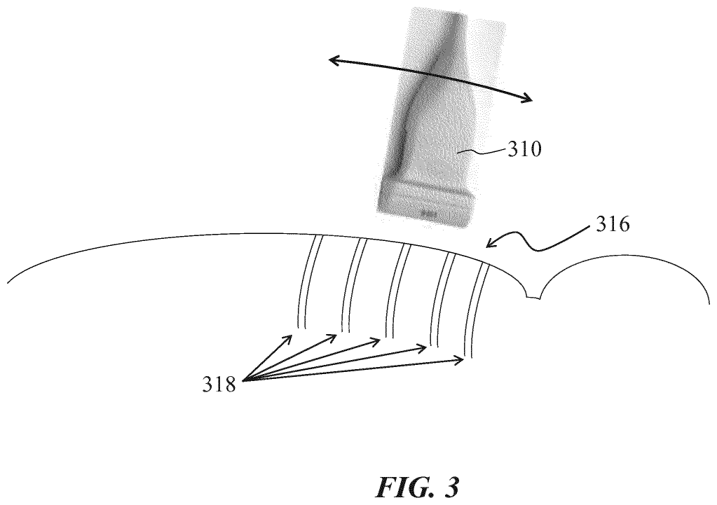

FIG. 3 is a representation of an ultrasound scan performed on a patient in accordance with principles of the present disclosure. In operation, the data acquisition unit or probe 310 containing an ultrasound sensor array may be moved over the surface of the chest region 316 of a patient to collect image data at multiple locations spanning one or both lungs. In some examples, the user may place the probe 310 longitudinally on the chest (in a head-to-toe orientation), as shown in FIG. 3. Automatic B-line detection can be initiated, for example upon receipt of a user input. The user may move the probe along the plane of imaging (in the direction of the arrows), carefully avoiding or minimizing any out-of-plane movement, during which the system may determine and update the B-line severity. The user can move the probe continuously or may pause at one or more locations to collect a series of image frames by acquiring echo signals 318 responsive to ultrasound pulses transmitted toward the target region 316. In this manner, image frames spanning at least one respiratory cycle (preferably two or more cycles if time permits), may be collected at each of a plurality of locations across the target region. The number of discrete locations may vary depending on the objectives of the user, the frequency setting of the ultrasound probe, and the clinical setting. For instance, in the ER/ICU setting, about 4 to about 6 locations may be examined, while internal medicine applications may involve a more thorough examination of about 25 to about 35 locations.

The distance covered by the probe 310 can be determined by a data processor communicatively coupled therewith, e.g., data processor 228, according to various techniques. For instance, the data processor can compute the distance traveled using image-based correlation techniques. In a particular embodiment, the probe can be moved longitudinally and the presence of one or more ribs identified, e.g., via shadowing. As the probe 310 is moved, the number of ribs traversed and the intercostal spacing therebetween can be identified and utilized by the data processor to estimate the total distance traveled. In addition or alternatively, image frames of the anatomical region above the pleural line can be used as a stationary reference point for frame-to-frame correlations to determine probe movement. As mentioned above with respect to FIG. 2, some embodiments may include a sensor, which may comprise an inertial sensor such as an accelerometer, configured to detect movement of the probe, such that image correlation performed by the data processor may be unnecessary or may be performed to substantiate the data acquired by the sensor. The sensor can also be configured to determine whether any out-of-plane movement of the probe 310 occurs during a particular scan, thereby ensuring that such movement is not included in the estimate of the total distance traveled. In some examples, a determination that out-of-plane movement occurred, especially substantial out-of-plane movement, may cause a notification to be communicated to the user, which may prompt the user to perform another scan. The probe 310 can be configured to obtain data from more than one spatial plane, either through spatial movement or via electronic steering implemented using a 2D array, for example.

After determining the distance traveled by the probe 310 to acquire the ultrasound data spanning the target region 316, the data processor can be configured to determine the spatial distribution of the B-lines identified across the target region. In some examples, the spatial distribution can be embodied in a B-line score, which may be specific to one or more intercostal spaces. For example, if the probe 310 covers a total of eight intercostal spaces, then eight B-line scores can be computed. The data processor can compare the eight B-line scores, for example to determine whether the scores are substantially similar. If the scores are similar, the processor may determine that the likelihood of cardiogenic pulmonary edema is high. If the scores are patchy, for example if there is a moderate to high number of B-lines within one intercostal space but not in another intercostal space, the processor may determine that the likelihood of non-cardiogenic pulmonary edema or focal disease, e.g., pneumonia, is high. In various embodiments, the B-line severity, embodied in a B-line score or otherwise, may be determined as a function of probe location during a particular scan, such that the severity may be updated one or more times as the probe 312 is moved across the target region. According to such embodiments, the user may input, on a user interface, the initial starting point of the transducer, e.g., the first intercostal space near the clavicle. The system may then compute the remainder of transducer locations, assuming probe movement is longitudinal. In some examples, the user can input the initial probe location as well as the movement direction, e.g., transverse (left-to-right across the chest) or longitudinal (head-to-toe). In addition or alternatively, the system may be configured to compile an overall B-line severity indication after a scan has been completed. The likelihood can be communicated to the user in the form of a numerical score in some examples, which may be displayed.

In some examples, the data processor may be configured to compare a B-line score, number and/or spatial distribution against a threshold. Scores above the threshold may indicate a moderate to high likelihood of cardiogenic pulmonary edema, and scores below the threshold may indicate a moderate to high likelihood of non-cardiogenic pulmonary edema. The threshold may be static or dynamic over time, and may be patient-specific. For example, a user may increase the threshold when examining a patient for which B-line scores have been higher than average during a previous scan which did not confirm the existence of cardiogenic pulmonary edema.

The display unit communicatively coupled with the probe can be configured to show the distribution of the detected B-lines and/or their severity along the path traversed by the probe on the patient's chest. The user interface 434 shown in FIG. 4A provides one example of a graphical representation that may be generated in accordance with the present disclosure. As shown, the user interface 434 can be configured to generate a graphical representation 440 of a chest/abdominal region of a patient. The representation 440 can be divided into a plurality of zones 442, which may span one or both lungs. The zones 442 shown in FIG. 4A are uniform and rectangular, but the size, shape and/or location of the zones may vary, along with the number of zones, which may range from 1 to 10, 20 or more. In some embodiments, the zones 442 may be customized by a user. That is, a user may specify the dimensions and/or location of one or more zones. In some examples, the zones 442 may be automatically displayed on the user interface 434, along with B-line statistics specific to each zone. A B-line score based on the number of B-lines present within a particular zone may be displayed within each zone. In some examples, one or more zones 442 may be colored to reflect the severity of B-lines present therein. For example, a high number of B-lines may be indicated by the color red, while a low number may be indicated by blue or green. In some embodiments, the color may be shown as a gradient distributed throughout the target region, enabling a more refined analysis of B-line “hotspots.” In addition or alternatively, B-line information determined during a scan may be displayed adjacent to the representation 440, for example in a table 444. In addition or alternatively, the user interface 434 may be configured to display at least one notification, such as notification 446a, indicating a “cardiac” cause of pulmonary edema, and/or notification 446b, indicating a “non-cardiac” cause of pulmonary edema. Such notification(s) can be displayed upon receiving an indication of the pulmonary edema etiology from the data processor communicatively coupled with the user interface.

FIG. 4B shows an ultrasound image 435 that may be displayed on the user interface 434 in some examples. As shown, lines or bars 448 may be overlaid onto confirmed B-lines, and a separate bar or line 450 may be overlaid onto the confirmed pleural line. In some embodiments, the lines 448, 450 can be displayed without the corresponding image, such that a user is presented only with a graphical representation or map of the B-lines and/or pleural line detected within a given ultrasound image. The thickness of the lines may correspond to the thickness and/or uniformity of the sonographic features they represent. For example, a strong B-line of uniform intensity extending a long distance from the pleural line may be assigned the highest weight. As further shown, an etiology notification 446a may be displayed in conjunction with a confidence metric 452 embodying the likelihood that the etiology determination is correct, as determined by a data processor in communication with the user interface 434. The confidence metric 452 may also embody the likelihood that a particular case of pulmonary edema is cardiogenic or non-cardiogenic. For instance, in the example shown, the confidence metric may indicate a 94% likelihood of cardiogenic pulmonary edema, which corresponds to a 6% likelihood of non-cardiogenic pulmonary edema. By displaying the ultrasound image 435, either with or without the bars, the user interface 434 allows a level of user interpretation, which can further substantiate the determinations made automatically by the system, thereby improving accuracy. In some examples, the user can toggle back-and-forth between the display shown on the user interface 434 in FIG. 4A and FIG. 4B, for instance. The display may update continuously as an ultrasound scan is performed, such that the notifications and/or bars change as the transducer used to acquire the image is moved.

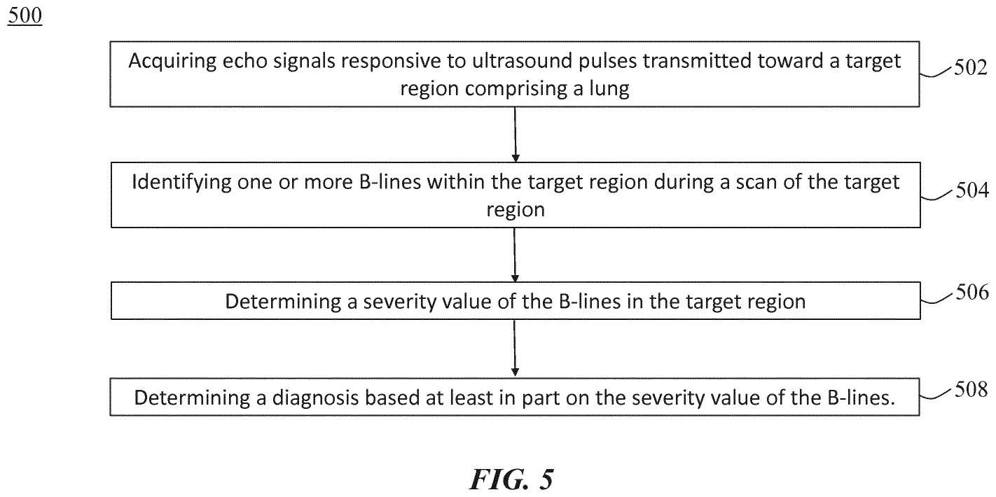

FIG. 5 is a block diagram of an ultrasound imaging method in accordance with the principles of the present disclosure. The example method 500 of FIG. 5 shows the steps that may be utilized, in any sequence, by the systems and/or apparatuses described herein for identifying and characterizing B-lines, and in some embodiments, determining the driving force of pulmonary edema in a patient. The method 500 may be performed by an ultrasound imaging system, such as system 500, or other systems including, for example, a mobile system such as LUMIFY by Koninklijke Philips N.V. (“Philips”). Additional example systems may include SPARQ and/or EPIQ, also produced by Philips.

In the embodiment shown, the method 500 begins at block 502 by “acquiring echo signals responsive to ultrasound pulses transmitted toward a target region comprising a lung.”

The method continues at block 504 by “identifying one or more B-lines within the target region during a scan of the target region.”

The method continues at block 506 by “determining a severity value of the B-lines in the target region.”

The method continues at block 508 by “determining a diagnosis based at least in part on the severity value of the B-lines.”

In various embodiments where components, systems and/or methods are implemented using a programmable device, such as a computer-based system or programmable logic, it should be appreciated that the above-described systems and methods can be implemented using any of various known or later developed programming languages, such as “C”, “C++”, “FORTRAN”, “Pascal”, “VHDL” and the like. Accordingly, various storage media, such as magnetic computer disks, optical disks, electronic memories and the like, can be prepared that can contain information that can direct a device, such as a computer, to implement the above-described systems and/or methods. Once an appropriate device has access to the information and programs contained on the storage media, the storage media can provide the information and programs to the device, thus enabling the device to perform functions of the systems and/or methods described herein. For example, if a computer disk containing appropriate materials, such as a source file, an object file, an executable file or the like, were provided to a computer, the computer could receive the information, appropriately configure itself and perform the functions of the various systems and methods outlined in the diagrams and flowcharts above to implement the various functions. That is, the computer could receive various portions of information from the disk relating to different elements of the above-described systems and/or methods, implement the individual systems and/or methods and coordinate the functions of the individual systems and/or methods described above.

In view of this disclosure it is noted that the various methods and devices described herein can be implemented in hardware, software and firmware. Further, the various methods and parameters are included by way of example only and not in any limiting sense. In view of this disclosure, those of ordinary skill in the art can implement the present teachings in determining their own techniques and needed equipment to affect these techniques, while remaining within the scope of the invention. The functionality of one or more of the processors described herein may be incorporated into a fewer number or a single processing unit (e.g., a CPU) and may be implemented using application specific integrated circuits (ASICs) or general purpose processing circuits which are programmed responsive to executable instruction to perform the functions described herein.

Although examples of the present system may have been described with particular reference to an ultrasound imaging system, it is also envisioned that the present system can be extended to other medical imaging systems where one or more images are obtained in a systematic manner. Accordingly, the present system may be used to obtain and/or record image information related to, but not limited to renal, testicular, breast, ovarian, uterine, thyroid, hepatic, lung, musculoskeletal, splenic, cardiac, arterial and vascular systems, as well as other imaging applications related to ultrasound-guided interventions. Further, the present system may also include one or more programs which may be used with conventional imaging systems so that they may provide features and advantages of the present system. Certain additional advantages and features of this disclosure may be apparent to those skilled in the art upon studying the disclosure, or may be experienced by persons employing the novel system and method of the present disclosure. Another advantage of the present systems and method may be that conventional medical image systems can be easily upgraded to incorporate the features and advantages of the present systems, devices, and methods.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

1. An ultrasound system comprising:
an ultrasound transducer configured to acquire echo signals responsive to ultrasound pulses transmitted toward a target region comprising a lung; and
one or more processors in communication with the ultrasound transducer, the processors configured to:
identify B-lines within the target region during a scan of the target region;
determine a severity value of the B-lines in the target region; and
determine a diagnosis based at least in part on the severity value of the B-lines.

2. The ultrasound system of claim 1, wherein the processors are configured to determine the severity value of the B-lines by determining a total number of B-lines.

3. The ultrasound system of claim 1, wherein the processors are configured to determine the severity value of the B-lines by determining a spatial distribution of the B-lines.

4. The ultrasound system of claim 3, wherein the processors are configured to determine the spatial distribution of the B-lines within one or more sub-regions of the target region.

5. The ultrasound system of claim 4, wherein each of the one or more sub-regions comprises an intercostal space such that a severity value is determined for each intercostal space within the target region.

6. The ultrasound system of claim 3, wherein the processors are configured to determine the spatial distribution by determining a distance covered by the ultrasound transducer during the scan of the target region and dividing the distance by a total number of B-lines identified.

7. The ultrasound system of claim 1, further comprising a graphical user interface configured to display an ultrasound image from at least one image frame generated from the ultrasound echoes.

8. The ultrasound system of claim 7, wherein the processors are further configured to cause the graphical user interface to display an annotated ultrasound image in which the B-lines are labeled.

9. The ultrasound system of claim 7, wherein the processors are further configured to cause the graphical user interface to display a graphical representation of the severity value of the B-lines in the target region.

10. The ultrasound system of claim 1, further comprising an inertial motion sensor configured to determine a distance covered by the ultrasound transducer during the scan of the target region.

11. The ultrasound system of claim 1, wherein the diagnosis comprises cardiogenic pulmonary edema or non-cardiogenic pulmonary edema.

12. The ultrasound imaging system of claim 11, wherein the processors are configured to distinguish cardiogenic pulmonary edema from non-cardiogenic pulmonary edema by applying a threshold to the severity value.

13. A method comprising:
acquiring echo signals responsive to ultrasound pulses transmitted toward a target region comprising a lung;
identifying B-lines within the target region during a scan of the target region;
determining a severity value of the B-lines in the target region; and
determining a diagnosis based at least in part on the severity value of the B-lines.

14. The method of claim 13, wherein determining the severity value of the B-lines comprises determining a total number of B-lines and/or a spatial distribution of the B-lines.

15. The method of claim 14, wherein determining the spatial distribution of the B-lines comprises determining a distance covered by the ultrasound transducer during the scan of the target region and dividing the distance by a total number of B-lines identified.

16. The method of claim 13, further comprising displaying an ultrasound image from at least one image frame generated from the ultrasound echoes.

17. The method of claim 16, further comprising displaying a graphical representation of the severity value of the B-lines in the target region and/or labeling the B-lines.

18. The method of claim 13, wherein the diagnosis comprises cardiogenic pulmonary edema or non-cardiogenic pulmonary edema.

19. The method of claim 18, further comprising distinguishing between cardiogenic pulmonary edema and non-cardiogenic pulmonary edema by applying a threshold to the severity value.

20. A non-transitory computer-readable medium comprising executable instructions, which when executed cause one or more processors to perform the method of claim 13.
